(19) United States
(12) Patent Application Publication
(10) Pub. No.: US 2005/0159769 A1
(43) Pub. Date: Jul. 21, 2005

(54) Balloon system and methods for treating obesity

(76) Inventor: John C Alverdy, Glenview, IL (US)

Correspondence Address: APPLIED MEDICAL RESOUCES CORPORATION, 22872 Avenida Empresa, Rancho Santa Margarita, CA, 92688, US

(21) Appl. No.: 10/513,583

(22) PCT Filed: Apr. 25, 2003
(86) PCT No.: PCT/US03/12782

Related U.S. Application Data
(60) Provisional application No. 60/379,540, filed on May 9, 2002.

(51) Int. Cl. A61M 29/00

(52) U.S. Cl. 606/192

(57) ABSTRACT

A medical system (100) for the treatment of morbid obesity comprising an inflatable balloon (110) implanted in a gastric cavity, a percutaneous fillant delivery tube (120) and a control module (130) connected to the tube for regulating the inflation and deflation of the balloon. The balloon may be individually contoured and inflated to occupy a large volume of the gastric cavity to provide a feeling of satiety. The balloon may also be deflated to give the gastric cavity lining a rest during less critical time.

This is a non-provisional application claiming priority to and incorporating by reference provisional application Ser. No. 60/379,540, filed May 9, 2002, entitled “Variably Cycled Percutaneous Endo-Balloon for Obesity.”

### BACKGROUND OF THE INVENTION

1. Field of the Invention

This invention generally relates to the treatment of morbid obesity and, more specifically, to a system and method for treating morbid obesity using a variably cycled percutaneous balloon implanted in the gastric cavity.

2. Discussion of the Prior Art

Morbid obesity is a major health problem confronting the general public and health care industry today. It is estimated that approximately 50% of the U.S. population is overweight and over ten million Americans are more than 100 pounds over their ideal weight. Generally, a person is considered morbidly (or seriously) obese if they are 100 pounds or more over their ideal weight. The morbidly obese group faces increased health risks including a higher likelihood of heart disease, hypertension, diabetes and certain cancers. Over 300,000 Americans die of obesity related illnesses each year. In addition, the morbidly obese generally have lower self-esteem and are more likely to suffer from depression than the general public.

Most obese individuals have struggled unsuccessfully with their weight for a lifetime. The numerous diets, behavioral therapy and treatments such as hypnosis, pituitary hormones and appetite suppressant drugs attest to the great difficulty many overweight people have in losing weight and keeping it off. Some of these weight loss strategies can be successful in the mildly obese people, but nearly all fail in individuals considered morbidly obese. These disappointing results have led many patients and their doctors to consider surgery as an option for weight loss.

Surgical techniques bring about weight loss primarily by limiting how much the stomach can hold. Today's most common surgical procedures to promote weight loss focus on decreasing food intake by restriction. Gastric banding, gastric bypass and vertical-banded gastroplasty are surgeries that limit the amount of food the stomach can hold by closing off or removing parts of the stomach. Other surgeries attempt to permanently fill the stomach with an inflated balloon. These treatments are invasive, require major surgery with hospitalization and are associated with complications.

The success rates of current treatments and procedures have been poor. With the restrictive procedure, the patient is usually limited to eating very small amounts of food at a time. For many people, this can create a “satisfied” feeling, but they often do not feel “full”. The ability to eat a large amount of food at one time is lost; consequently, many patients return to eating excessive amounts of high calorie or high sugar liquid foods. Essentially, their diet includes milk shakes and ice cream.

As to the balloon procedure of the past, very limited positive results were achieved. The balloon was relatively small when compared to the overall volume of the morbidly obese stomach. This is due to physiological limitation on the balloon volume. That is, complications of the device precluded enlarging it to a volume that would occupy more of the stomach. Yet, in order for the balloon to achieve a patient's feeling of fullness and satiation, the balloon would need to occupy a large portion (volume) of the patient's stomach. A balloon occupying this much volume without fixation or an inflation/deflation cycling has the potential of blocking food flow and causing necrosis of the stomach wall, ulcers and/or bleeding.

Moreover, success depends on the ability of a treatment to “normalize” not only the mechanical and neurohormonal sensation of feeling full and satiated, but also involves psychological factors. Both the mechanical and neurohormonal factors relate to one's need to feel “full” and “satiated”. Chemicals released by the stomach during the digestive process largely drive these factors. In other words, filling the stomach or limiting its pouch size controls these chemicals. Current surgical approaches, however, fail to achieve this global feeling of “satiety” response as they restrict food entry only into the small proximal stomach pouch and bypass the distal stomach where most of the neurohormonal chemical are normally released. Medical therapy is focused almost exclusively at the brain level and is likely to continue to fail as patients experience mood disorders and complications from medications. Accordingly, there is a need for a system and method for treating morbid obesity by restoring or normalizing the appropriate “fullness signals” from the stomach itself as this is the organ that regulates fullness. In particular, the system and method of the invention should cause a feeling of satiety from the stomach itself with less consumption of food by a morbidly obese patient.

### SUMMARY OF THE INVENTION

A system and method for treating morbid obesity using a variably cycled percutaneous balloon implanted in the gastric cavity to elicit signals directly from the entire stomach in order to cause a feeling of satiety with less food. This novel approach has the potential to offer a less invasive, more complete elicitation of the feeling of fullness in patients who chronically, and perhaps genetically overeat. The system of the invention includes a balloon device that is contoured to occupy the vast majority of the volume of the stomach. The system also has the capacity to automatically inflate and deflate the balloon, thereby avoiding the problem of pressure induced injury. With the advent of CT scanning and 3-dimensional imaging, patients may have balloons individually designed to meet the specific morphologic features of their stomachs. By fixation of the balloon device, the problems of migration and obstruction are avoided. Furthermore, the system and process of the invention apply appropriate inflation/deflation cycling with a computerized device so as to avoid complications of past devices.

These and other features and advantages of the invention will become more apparent with a discussion of preferred embodiments in reference to the associated drawings.

### DESCRIPTION OF THE DRAWINGS

FIG. 1 illustrates a schematic view of a variably cycled percutaneous balloon placed within the gastric cavity of an individual in accordance with an embodiment of the invention; and

FIG. 2 illustrates a cross-sectional view of an inflatable balloon and a fillant delivery tube according to the present invention.

### DESCRIPTION OF PREFERRED EMBODIMENTS AND BEST MODE OF THE INVENTION

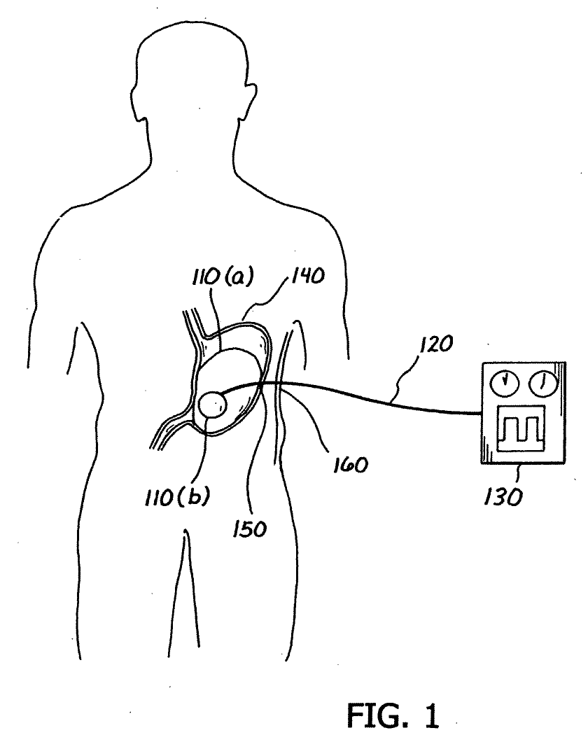

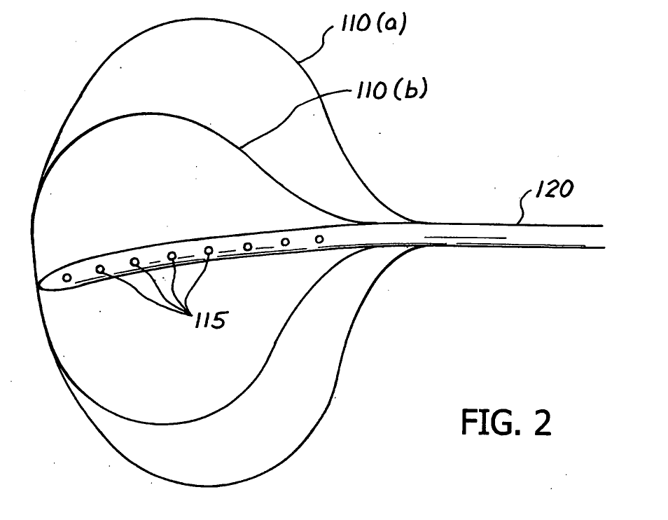

With reference to FIGS. 1 and 2, a variably cycled percutaneous balloon system 100 for treating morbid obesity is illustrated and comprises an inflatable balloon 110 individually contoured to each patient's stomach, a percutaneous inflation or fillant delivery tube 120 having a proximal end and a distal end connected to the balloon 110, and a control module 130 connected to the proximal end of the tube 120. The tube 120 includes at least one opening 115 for filling the balloon 110 with a biocompatible fillant. The control module 130 variably controls the inflation and deflation of the balloon 110 with the biocompatible fillant such as a liquid, gas, gel or a mixture thereof. In accordance with the teachings of the present invention, the tube 120 is passed through and affixed to abdominal wall 160 and stomach wall 150. The balloon 110 is then positioned into the stomach or gastric cavity 140. The positioning of the balloon 110 may be done, e.g., by the percutaneous endoscopic gastrostomy (PEG) technique, which is known in the art. The balloon 110 and tube 120 may be separate or integral components that are constructed from any surgical grade material. For example, the balloon 110 may be made from latex rubber which expands upon introduction of a fillant, and the tube 120 may be constructed of a metal or plastic material. The tube 120 is connected to the control module 130, which may be a fixed unit or a portable unit mounted to the patient's side. The control module 130 may be a personal computer such as a desktop computer, a laptop computer or a handheld computer. The control module 130 further includes a device such as a pump for introducing and removing a fillant to and from the balloon 110.

A novel feature of the system 100 is it variably controls the inflation and deflation of the balloon 110. For example, the system 100 may inflate and deflate the balloon 110 throughout a predetermined period of time such as a 24-hour period. The balloon 110 would occupy a large volume of the stomach 140 (as shown by reference number 110(a)) when it would be most beneficial for weight loss, and deflate to give the stomach lining a rest (as shown by reference number 110(b)) during less critical time, e.g., during sleeping time. Furthermore, an algorithm tailored to each patient's needs and programmed into the control module 130 is used to control the balloon size to minimize the desire to eat and to prevent blockage or stomach lining necrosis. Unlike the restrictive procedures of the prior art, the variable inflated balloon 110 would not limit nutrient absorption and not lead to altered food choices. This is achieved as the balloon 110 contacts a major portion of the stomach wall 150 when the balloon 110 is fully inflated. Thus, the system 100 of the invention creates a feeling of fullness and satiation by balancing the physiological, neurohormonal and chemical factors.

It will be understood that many modifications can be made to the disclosed embodiments without departing from the spirit and scope of the invention. As such, the above description should not be construed as limiting the invention, but should be interpreted as merely exemplary of preferred embodiments.

1. A medical system for the treatment of morbid obesity, comprising:
an inflatable balloon implanted in a gastric cavity;
a percutaneous fillant delivery tube having a proximal end a distal end connected to the balloon, said tube being passed through and affixed to an abdominal wall and the gastric cavity; and
a control module connected to the proximal end of the tube for regulating the inflation and deflation of the balloon.

2. The medical system of claim 1, wherein the balloon is individually contoured to the gastric cavity.

3. The medical system of claim 1, wherein the balloon is inflated with a biocompatible fillant of at least one of a liquid, a gas and a gel.

4. The medical system of claim 1, wherein the control module may be a fixed unit or a portable unit.

5. The medical system of claim 1, wherein the control module is a desktop computer.

6. The medical system of claim 1, wherein the control module is a laptop computer or a handheld computer.

7. The medical system of claim 1, wherein the control module is programmed to inflate and deflate the balloon over a predetermined period of time.

8. The medical system of claim 1, wherein the balloon inflates to occupy a large volume of the gastric cavity to provide a feeling of satiety.

9. The medical system of claim 1, wherein the balloon deflates to give the gastric cavity lining a rest during less critical time.

10. The medical system of claim 1, wherein the control module further comprises a pump for introducing and removing a fillant to and from the balloon.

11. The medical system of claim 2, wherein the individually contoured balloon is configured to expand to a specific shape.

12. The medical system of claim 1, wherein the balloon and the tube are integral.

13. A method for treating morbid obesity of a patient with an abdominal wall and a gastric cavity, comprising the steps of:
performing a percutaneous endoscopic gastrostomy to implant a balloon through the abdominal wall and into the gastric cavity;
affixing a percutaneous fillant delivery tube to the abdominal wall and the gastric cavity, said tube having a proximal end and a distal end connected to the balloon;
connecting the proximal end of the tube to an external control module; and
regulating the inflation and deflation of the balloon with a fillant using the control module.

14. The medical system of claim 13, further comprising the step of programming the control module to inflate and deflate the balloon over a predetermined period of time.

15. The medical system of claim 13, further comprising the step of inflating the balloon to occupy a large portion of the gastric cavity to provide a feeling of satiety.

16. The medical system of claim 13, further comprising the step of deflating the balloon to give the gastric cavity lining a rest during less critical time.

17. The medical system of claim 13, wherein the balloon is individually contoured to the gastric cavity.

18. The medical system of claim 13, wherein the fillant is biocompatible and is at least one of a liquid, a gas and a gel.

19. The medical system of claim 13, wherein the control module may be a fixed unit or a portable unit.

20. The medical system of claim 19, wherein the control module further comprises a pump for introducing and removing the fillant to and from the balloon.

21. The medical system of claim 17, wherein the individually contoured balloon is configured to expand to a specific shape.
